(19) United States
(12) Patent Application Publication
(10) Pub. No.: US 2023/0293632 A1
(43) Pub. Date: Sep. 21, 2023

(54) USE OF PERLECAN AND FRAGMENTS THEREOF TO REDUCE THE RISK OF DEATH IN STROKE PATIENTS

(71) Applicant: STREAM BIOMEDICAL, INC., Houston, TX (US)

(72) Inventors: Huston Davis ADKISSON, St. Louis, MO (US); Bryan Lloyd CLOSSEN, Stafford, TX (US); Gary B. GAGE, Missouri City, TX (US)

(73) Assignee: STREAM BIOMEDICAL, INC., Houston, TX (US)

(21) Appl. No.: 18/017,669

(22) PCT Filed: Jul. 26, 2021
(86) PCT No.: PCT/US2021/070948

§ 371 Date: Jan. 23, 2023

Related U.S. Application Data
(60) Provisional application No. 63/056,059, filed on Jul. 24, 2020.
(60) Provisional application No. 63/061,308, filed on Aug. 5, 2020.

(51) Int. Cl. A61K 38/17; A61K 38/49; A61P 9/10

(52) U.S. Cl. CPC A61K 38/1709; A61K 38/49; A61P 9/10

(57) ABSTRACT

The present disclosure is directed to the use of perlecan compositions to reduce the risk of mortality in subjects due to neurological injury such as stroke, including large vessel occlusion, and traumatic brain injury. The disclosure is also directed to the use of perlecan compositions to reduce mortality in stroke patients treated with tPA.

### PRIORITY CLAIM

This application claims benefit of priority to U.S. Provisional Application Ser. No. 63/056,059, filed Jul. 24, 2020, and 63/061,308, filed Aug. 5, 2020, the entire contents of both applications being hereby incorporated by reference.

Pursuant to 37 C.F.R. 1.821(c), a sequence listing is submitted herewith as an ASCII compliant text file named “STRMP0003WO_ST25.txt”, created on Jul. 26, 2021 and having a size of ˜39 kilobytes. The content of the aforementioned file is hereby incorporated by reference in its entirety.

### BACKGROUND

### 1. Field of the Disclosure

The present disclosure relates generally to the fields of medicine, vascular disease, and neurobiology. More particular, the disclosure relates to use of perlecan and fragments thereof to reduce the risk of death in patients suffering strokes, and in particular those suffering from ischemic stroke characterized by large vessel occlusion of the anterior cerebral vasculature.

### 2. Background

Every year, more than 795,000 people in the United States have a stroke with about 140,000 of these subjects dying, accounting for 1 out of every 20 deaths annually. In fact, someone in the United States has a stroke every 40 second and every 4 minutes, someone dies of stroke. Stroke costs the United States an estimated $34 billion each year, including the cost of health care services, medicines to treat stroke, and missed days of work. Stroke is also a leading cause of serious long-term disability, reducing mobility in more than half of stroke survivors age 65 and over.

About 87% of all strokes are ischemic strokes, in which blood flow to the brain is blocked. In contrast, hemorrhagic strokes result from bleeding. Treatment options for strokes are limited and some of the most commonly used treatments—aspirin and tissue plasminogen activator—need to be employed early in the event or they can cause additional damage. Thus, improved methods to treat strokes and reduce the significant risk of death that strokes present, particularly those outside the “early” window for intervention, are desperately needed.

### SUMMARY

Thus, in accordance with the present disclosure, there is provided a method of reducing the risk of mortality in a subject suffering from or at risk of a neurological injury comprising administering to said subject a composition comprising perlecan domain V or a functional fragment thereof, wherein the condition is not a neurodegenerative disease. The functional fragment may be LG3. The subject may be suffering from a stroke or is at risk of a stroke, such as because said subject has previously suffered from a stroke, has atrial fibrillation, has diabetes, has hypertension, has cancer or has a viral infection, such as SARS-Cov-2 or a variant thereof. The stroke may be an ischemic stroke, such as due to a large vessel occlusion. The large vessel occlusion may be located in the anterior cerebral vasculature. The stroke may be a hemorrhagic stroke. Alternatively, the subject may be suffering from a traumatic brain injury, such as caused by a concussive blast or blunt impact trauma.

Administering may comprise intraperitoneal administration, intravenous administration, intra-arterial administration, intrathecal administration, intracranial administration, intraosseous administration, intramuscular administration, subcutaneous administration, or oral (buccal) administration. The subject may have received or may be receiving a thrombolytic therapy, such as tissue plasminogen activator. The composition may be administered more than once, such as 2, 3, 4, 5, 6, 7, 8, 9 or 10 times. The composition may be administered twice daily (such as every 12 hours), daily, every other day, every third day, twice a week, weekly, every other week, twice monthly or monthly. The method may further comprise administering to a second anti-stroke therapy, such as other than a thrombolytic therapy.

Also provided is a method of reducing the risk of mortality in a subject suffering from a stroke and receiving a thrombolytic or embolytic therapy comprising administering to said subject perlecan domain V or a functional fragment thereof. The functional fragment may be LG3. The stroke may be an ischemic stroke, such as due to a large vessel occlusion. The large vessel occlusion may be located in the anterior cerebral vasculature. The stroke may be a hemorrhagic stroke.

Administering may comprise intraperitoneal administration, intravenous administration, intra-arterial administration, intrathecal administration, intracranial administration, intraosseous administration, intramuscular administration, subcutaneous administration, or oral (buccal) administration. The thrombolytic therapy may be tissue plasminogen activator. The composition may be administered more than once, such as 2, 3, 4, 5, 6, 7, 8, 9 or 10 times. The composition may be administered twice daily (such as every 12 hours), daily, every other day, every third day, twice a week, weekly, every other week, twice monthly or monthly. The method may further comprise administering to a second anti-stroke therapy other than a thrombolytic/embolytic therapy. The thrombolytic/embolytic therapy may be administered within three hours of said stroke occurring or administered more than three hours after said stroke occurring. This effectively serves to extend the window for use of thrombolytics/embolytics in stroke therapy.

In another embodiment, there is provided method of reducing the risk of mortality in a subject suffering from a stroke comprising treating to said subject with (a) perlecan domain V or a functional fragment thereof and (b) thrombectomy or embolectomy. The functional fragment may be LG3. The stroke may be an ischemic stroke, such as due to a large vessel occlusion. The large vessel occlusion may be located in the anterior cerebral vasculature. The stroke may be a hemorrhagic stroke. Administering may comprise intraperitoneal administration, intravenous administration, intra-arterial administration, intrathecal administration, intracranial administration, intraosseous administration, intramuscular administration, subcutaneous administration, or oral (buccal) administration.

The perlecan domain V or a functional fragment thereof may be administered more than once, such as 2, 3, 4, 5, 6, 7, 8, 9 or 10 times. The perlecan domain V or a functional fragment may be administered twice daily (such as every 12 hours), daily, every other day, every third day, twice a week, weekly, every other week, twice monthly or monthly. The method may further comprise administering a further anti-stroke therapy, such as other than thrombectomy/embolectomy. The thrombectomy/embolectomy may be performed within three hours of said stroke occurring. The thrombectomy/embolectomy therapy may be performed more than three hours after said stroke occurring. This effectively serves to extend the window for use of thrombectomy/embolectomy in stroke therapy.

Also provided is a method of treating, preserving, or stabilizing a transplant material comprising bathing, incubating, infusing, injecting or otherwise contacting the material with (a) perlecan domain V or a functional fragment thereof.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-D. LG3 Protection from tPA-induced Post-Stroke Hemorrhage: Gross necropsy of 1/10 LG3/tPA coadministration study at post-stroke day 3 revealed a massive hemorrhage/bleed from the MCA which escaped the cranium and coagulated in and around the brain and skull. TTC revealed the brain was protected from injury from the bleed and stroke, with lesion size measured within expected values for an LG3-treated mouse undergoing 60-minute CCA-MCA occlusion. Dosing at 6 mg/kg.

FIG. 2. Change in Body Mass Following tMCAO: Body weights were recorded daily for 7 days following 60-minute MCA occlusion via intraluminal filament. LG3 treatment resulted in significantly less weight lost relative to vehicle-treated controls and accelerated acute body weight recovery following stroke. Dosing at 6 mg/kg.

FIG. 3. Kaplan-Meier Plot for Mortality Assessment: Survival data was recorded for the experiments described in FIG. 2. Analysis revealed LG3-treatment resulted in significantly improved survival relative to vehicle-treated controls. Dosing at 6 mg/kg.

### DESCRIPTION OF ILLUSTRATIVE EMBODIMENTS

As discussed above, stroke is a significant cause of mortality in the United States and this is also true for the world in general. Moreover, it places considerable strain on medical providers and hospitals given the need for supportive care. The costs for treating stroke victims in both the acute and recovery phases are staggering.

Here, the inventors have determined that in addition to the previously known neuroprotective effects of perlecan domain V (PDV) and its fragment, LG3, these agents can also protect patients against catastrophic events associated with stroke that would otherwise likely cause death. This new finding suggests that the application of PDV and LG3 in the appropriate patients may do much more than simply limit the damage to brain tissue during and after stroke, but also substantially reduce the risk of death from major ischemic and/or hemorrhagic events.

It is important to note that human recombinant DV has already been shown to be well-tolerated and non-toxic in animal models of disease and specifically homes to pathologic tissue (Bix et al., J. Natl. Cancer Inst. 98.22 (2006): 1634-46). These results can be explained by the fact that DV is a naturally occurring protein fragment readily found in blood and CSF proteomes (Bix & Iozzo, Microsc. Res. Tech. 71.5 (2008): 339-48). Previous experiments have also shown DV and LG3 can cross the blood brain barrier.

These and other aspects of the disclosure are described in detail below.

### I. Neurological Injury

A. Stroke and Related Brain Injury

A stroke is a medical condition in which poor blood flow to the brain causes cell death. There are two main types of stroke: ischemic, due to lack of blood flow, and hemorrhagic, due to bleeding. Both cause parts of the brain to stop functioning properly. Signs and symptoms of a stroke may include an inability to move or feel on one side of the body, problems understanding or speaking, dizziness, or loss of vision to one side. Signs and symptoms often appear soon after the stroke has occurred. If symptoms last less than one or two hours, the stroke is a transient ischemic attack (TIA), also called a mini-stroke. A hemorrhagic stroke may also be associated with a severe headache. The symptoms of a stroke can be permanent. Long-term complications may include pneumonia and loss of bladder control.

The main risk factor for stroke is high blood pressure. Other risk factors include tobacco smoking, obesity, high blood cholesterol, diabetes mellitus, a previous TIA, end-stage kidney disease, and atrial fibrillation. An ischemic stroke is typically caused by blockage of a blood vessel, though there are also less common causes. A hemorrhagic stroke is caused by either bleeding directly into the brain or into the space between the brain's membranes. Bleeding may occur due to a ruptured brain aneurysm. Diagnosis is typically based on a physical exam and supported by medical imaging such as a CT scan or MRI scan. A CT scan can rule out bleeding, but may not necessarily rule out ischemia, which early on typically does not show up on a CT scan. Other tests such as an electrocardiogram (ECG) and blood tests are done to determine risk factors and rule out other possible causes. Low blood sugar may cause similar symptoms.

Prevention includes decreasing risk factors, surgery to open up the arteries to the brain in those with problematic carotid narrowing, and warfarin in people with atrial fibrillation. Aspirin or statins may be recommended by physicians for prevention. A stroke or TIA often requires emergency care. An ischemic stroke, if detected within three to four and half hours, may be treatable with a medication that can break down the clot. Some hemorrhagic strokes benefit from surgery. Treatment to attempt recovery of lost function is called stroke rehabilitation, and ideally takes place in a stroke unit; however, these are not available in much of the world.

In 2013 approximately 6.9 million people had an ischemic stroke and 3.4 million people had a hemorrhagic stroke. In 2015 there were about 42.4 million people who had previously had a stroke and were still alive. Between 1990 and 2010 the number of strokes which occurred each year decreased by approximately 10% in the developed world and increased by 10% in the developing world. In 2015, stroke was the second most frequent cause of death after coronary artery disease, accounting for 6.3 million deaths (11% of the total). About 3.0 million deaths resulted from ischemic stroke while 3.3 million deaths resulted from hemorrhagic stroke. About half of people who have had a stroke live less than one year. Overall, two thirds of strokes occurred in those over 65 years old.

Strokes can be classified into two major categories: ischemic and hemorrhagic. Ischemic strokes are caused by interruption of the blood supply to the brain, while hemorrhagic strokes result from the rupture of a blood vessel or an abnormal vascular structure. About 87% of strokes are ischemic, the rest being hemorrhagic. Bleeding can develop inside areas of ischemia, a condition known as “hemorrhagic transformation.” It is unknown how many hemorrhagic strokes actually start as ischemic strokes.

In an ischemic stroke, blood supply to part of the brain is decreased, leading to dysfunction of the brain tissue in that area. There are four reasons why this might happen:
Thrombosis (obstruction of a blood vessel by a blood clot forming locally)
Embolism (obstruction due to an embolus from elsewhere in the body)
Systemic hypoperfusion (general decrease in blood supply, e.g., in shock)
Cerebral venous sinus thrombosis.
   A stroke without an obvious explanation is termed cryptogenic (of unknown origin); this constitutes 30-40% of all ischemic strokes.

There are various classification systems for acute ischemic stroke. The Oxford Community Stroke Project classification (OCSP, also known as the Bamford or Oxford classification) relies primarily on the initial symptoms: based on the extent of the symptoms, the stroke episode is classified as total anterior circulation infarct (TACI), partial anterior circulation infarct (PACT), lacunar infarct (LACI) or posterior circulation infarct (POCI). These four entities predict the extent of the stroke, the area of the brain that is affected, the underlying cause, and the prognosis. The TOAST (Trial of Org 10172 in Acute Stroke Treatment) classification is based on clinical symptoms as well as results of further investigations: on this basis, a stroke is classified as being due to (1) thrombosis or embolism due to atherosclerosis of a large artery, (2) an embolism originating in the heart, (3) complete blockage of a small blood vessel, (4) other determined cause, (5) undetermined cause (two possible causes, no cause identified, or incomplete investigation). Users of stimulants such as cocaine and methamphetamine are at a high risk for ischemic strokes.

There are two main types of hemorrhagic stroke. The first is intracerebral hemorrhage, which is basically bleeding within the brain itself (when an artery in the brain bursts, flooding the surrounding tissue with blood), due to either intraparenchymal hemorrhage (bleeding within the brain tissue) or intraventricular hemorrhage (bleeding within the brain's ventricular system). The second is subarachnoid hemorrhage, which is basically bleeding that occurs outside of the brain tissue but still within the skull, and precisely between the arachnoid mater and pia mater (the delicate innermost layer of the three layers of the meninges that surround the brain).

These two main types of hemorrhagic stroke are also two different forms of intracranial hemorrhage, which is the accumulation of blood anywhere within the cranial vault; but the other forms of intracranial hemorrhage, such as epidural hematoma (bleeding between the skull and the dura mater, which is the thick outermost layer of the meninges that surround the brain) and subdural hematoma (bleeding in the subdural space), are not considered “hemorrhagic strokes.” Hemorrhagic strokes may occur on the background of alterations to the blood vessels in the brain, such as cerebral amyloid angiopathy, cerebral arteriovenous malformation and an intracranial aneurysm, which can cause intraparenchymal or subarachnoid hemorrhage.

In addition to neurological impairment, hemorrhagic strokes usually cause specific symptoms (for instance, subarachnoid hemorrhage classically causes a severe headache known as a thunderclap headache) or reveal evidence of a previous head injury. Stroke symptoms typically start suddenly, over seconds to minutes, and in most cases do not progress further. The symptoms depend on the area of the brain affected. The more extensive the area of the brain affected, the more functions that are likely to be lost. Some forms of stroke can cause additional symptoms. For example, in intracranial hemorrhage, the affected area may compress other structures. Most forms of stroke are not associated with a headache, apart from subarachnoid hemorrhage and cerebral venous thrombosis and occasionally intracerebral hemorrhage.

Stroke symptoms typically start suddenly, over seconds to minutes, and in most cases do not progress further. The symptoms depend on the area of the brain affected. The more extensive the area of the brain affected, the more functions that are likely to be lost. Some forms of stroke can cause additional symptoms. For example, in intracranial hemorrhage, the affected area may compress other structures. Most forms of stroke are not associated with a headache, apart from subarachnoid hemorrhage and cerebral venous thrombosis and occasionally intracerebral hemorrhage.

For ischemic stroke, aspirin reduces the overall risk of recurrence by 13% with greater benefit early on. Definitive therapy within the first few hours is aimed at removing the blockage by breaking the clot down (thrombolysis), or by removing it mechanically (thrombectomy). Tight blood sugar control in the first few hours does not improve outcomes and may cause harm. High blood pressure is also not typically lowered as this has not been found to be helpful. Cerebrolysin, a mix of pig brain tissue used to treat acute ischemic stroke in many Asian and European countries, does not improve outcomes and may increase the risk of severe adverse events.

Thrombolysis, such as with recombinant tissue plasminogen activator (rtPA, tPA), in acute ischemic stroke, when given within three hours of symptom onset, results in an overall benefit of 10% with respect to living without disability. It does not, however, improve chances of survival. Benefit is greater the earlier it is used. Between three and four and a half hours the effects are less clear. The AHA/ASA recommend it for certain people in this time frame. After four and a half hours thrombolysis worsens outcomes. These benefits or lack of benefits occurred regardless of the age of the person treated. There is no reliable way to determine who will have an intracranial bleed post-treatment versus who will not. In those with findings of savable tissue on medical imaging between 4.5 hours and 9 hours or who wake up with a stroke, alteplase (tPA) results in some benefit. tPA is endorsed by the American Heart Association, the American College of Emergency Physicians and the American Academy of Neurology as the recommended treatment for acute stroke within three hours of onset of symptoms as long as there are no other contraindications (such as abnormal lab values, high blood pressure, or recent surgery). Intra-arterial fibrinolysis, where a catheter is passed up an artery into the brain and the medication is injected at the site of thrombosis, has been found to improve outcomes in people with acute ischemic stroke.

Mechanical removal of the blood clot causing the ischemic stroke, called mechanical thrombectomy, is a potential treatment for occlusion of a large artery, such as the middle cerebral artery. In 2015, one review demonstrated the safety and efficacy of this procedure if performed within 12 hours of the onset of symptoms. It did not change the risk of death, but reduced disability compared to the use of intravenous thrombolysis which is generally used in people evaluated for mechanical thrombectomy. Certain cases may benefit from thrombectomy up to 24 hours after the onset of symptoms.

Ischemic strokes affecting large portions of the brain can cause significant brain swelling with secondary brain injury in surrounding tissue. This phenomenon is mainly encountered in strokes affecting brain tissue dependent upon the middle cerebral artery for blood supply and is also called “malignant cerebral infarction” because it carries a dismal prognosis. Relief of the pressure may be attempted with medication, but some require hemicraniectomy, the temporary surgical removal of the skull on one side of the head. This decreases the risk of death, although some people—who would otherwise have died—survive with disability.

For hemorrhagic stroke, people with intracerebral hemorrhage require supportive care, including blood pressure control if required. People are monitored for changes in the level of consciousness, and their blood sugar and oxygenation are kept at optimum levels. Anticoagulants and anti-thrombotics can make bleeding worse and are generally discontinued (and reversed if possible). A proportion may benefit from neurosurgical intervention to remove the blood and treat the underlying cause, but this depends on the location and the size of the hemorrhage as well as patient-related factors, and ongoing research is being conducted into the question as to which people with intracerebral hemorrhage may benefit.

In subarachnoid hemorrhage, early treatment for underlying cerebral aneurysms may reduce the risk of further hemorrhages. Depending on the site of the aneurysm this may be by surgery that involves opening the skull or endovascularly (through the blood vessels).

B. Traumatic Brain Injury

Traumatic brain injury usually results from a violent blow or jolt to the head or body. TBI can also be caused by an object that penetrates brain tissue, such as a bullet or shattered piece of skull that ultimately leads to brain cell death. Common events that cause TBI include falls, vehicle related collisions, violence, sports injuries, explosive blast or other combat injuries. TBI can result in bruising, torn tissues, bleeding and other physical damage to the brain, which could lead to long-term complications or death. Symptoms could include loss of consciousness, repeated vomiting, seizures, loss of coordination, profound confusion, slurred speech. Research studies have suggested that repeated or severe TBI might increase the risk of neurodegenerative diseases, including Alzheimer's disease/AD and Parkinson's disease/PD.

### II. Large Vessel Occlusion

As noted above, ischemic strokes are responsible for over 80% of all strokes. A particular category of ischemic stroke results from occlusion of large vessels, hence termed “large vessel occlusion” or LVO, have a significantly higher morbidity and mortality rates than small vessel-related strokes. Furthermore, treatments tend to be less effective with patients exhibiting LVO due to larger clot burden. Specialized endovascular therapies that remove clots have been used for LVO but can be challenging, time-consuming and not without risk. Thus, LVO is a particularly significant application for the compositions and methods disclosed herein.

Four mechanisms of acute LVO have been identified. The first is in situ occlusion due to atheromatous plaque rupture of an intracranial artery. The second is an artery-to-artery embolism in which embolic fragments arise from extracranial arteries affected by stenosis, ulceration with plague rupture, or dissection. The third is an embolism originating from the heart, often caused by atrial fibrillation. And fourth, there are LVO's of unknown cause (“cryptogenic”), suspected to arise form occult paroxysmal atrial fibrillation. The incidence of LVO from these four mechanisms varies.

One type of LVO is anterior cerebral artery syndrome, a condition whereby the blood supply from the anterior cerebral artery (ACA) is restricted, leading to a reduction of the function of the portions of the brain supplied by that vessel: the medial aspects of the frontal and parietal lobes, basal ganglia, anterior fornix and anterior corpus callosum. Depending upon the area and severity of the occlusion, signs and symptoms may vary within the population affected with ACA syndrome. Blockages to the proximal (A1) segment of the vessel produce only minor deficits due to the collateral blood flow from the opposite hemisphere via the anterior communicating artery. Occlusions distal to this segment will result in more severe presentation of ACA syndrome. Contralateral hemiparesis and hemi sensory loss of the lower extremity is the most common symptom associated with ACA syndrome. LVO/ACA is another significant application for the compositions and methods disclosed herein.

### III. Perlecan and Domain V (DV)

The amino acid sequence of human perlecan protein has been assigned Swiss-Prot accession number P98160 (SEQ ID NO: 1). The amino acid sequence of residues 3658 to 4391 constitutes Domain V (DV). Several naturally occurring variants are listed for P98160 in the Swiss-Prot database including an S-to-N amino acid change at position 4331. The inventors have demonstrated that N to Q substitutions at positions 3780, 3836 and 4068, not found in natural full length DV, have therapeutic potential. Thus, de-glycosylated mutants of DV and LG3 are disclosed as well. DV is subdivided into the following regions: residues 3663-3843 (181 amino acids) Laminin G-like domain 1 (LG1); residues 3844-3881 (38 amino acids) EGF-like domain 1; residues 3884-3922 (39 residues) EGF-like domain 2, residues 3928-4103 (176 amino acids), Laminin G-like domain 2 (LG2); residues 4104-4141 (38 amino acids) EGF-like domain 3; residues 4143-4176 (34 amino acids) EGF-like domain 4; residues 4197-4391 (189 amino acids) Laminin G-like domain 3 (LG3). There is a natural BMP-1 cleavage site between residues 4196 and 4197, which generates a peptide of residues 4197-4391, i.e., the LG3 domain. These delineations between domains are based on the annotations in the Swiss-Prot database for P98160. Several species variants of the human sequences are known. For example, mouse, Drosophila and chicken perlecan are assigned Swiss Prot accession numbers Q05793, Q8MPN3 and Q6 KD71-Chik, respectively; Macaca mulatta (rhesus monkey), Equus caballus (horse), Bos taurus (cow), and Danio rerio (zebrafish) perlecan sequences have also been identified.

The disclosure provides agents comprising or consisting of fragments of perlecan containing a contiguous segment of residues from within DV and more particularly from within the laminin-G-like domain 3 region of DV (LG3). Some such agents contain a segment of at least 5, 6, 7, 8, 9, 10, 11, 12, 13, 14, 15, 20, 50, 100, 150, 193, 200, 300, 400, 500, 600, 700 or 728 contiguous residues of within DV and/or, in the case of contiguous fragments or segments of 193 residues within LG3. Some agents contain no more or no less than 700, 600, 500, 400, 300, or 200 contiguous residues of DV or LG3. For example, some agents have 5-728, 5-500, or 5-193 contiguous amino acids from DV or LG3, in particular wholly or partly within LG3. Some agents have 193-728, 193-500, 193-200, 10-100 or about 193 contiguous amino acids from DV, in particular wholly or partly within LG3.

Some exemplary agents include agents comprising or consisting of perlecan peptides defined by any of the following amino acid coordinates: 4197-4389, 4197-4390, 4197-4391, 4198-4389, 4198-4390, 4198-4391, 4199-4389, 4199-4390, 4199-4391, 4200-4389, 4200-4390, 4200-4391, 4201-4389, 4201-4390, 4201-4391, 4202-4389, 4202-4390, 4202-4391. Reference to the peptide 4197-4391, for example, means the peptide beginning at residue 4197 and ending at residue 4391 and including all residues of the LG3 of DV. An agent comprising such a peptide may include additional contiguous flanking residues from DV. Other peptides are similarly defined. The disclosure also provides peptides that differ from any of the peptides mentioned above by up to 5, 10, 15, 20, 25 or 50 deletions, additions or substitutions of amino acids. Such deletions, additions or substitutions can be internal or at the C- or N-terminus. Substitutions can be conservative or nonconservative. Additions can include additional contiguous residues from DV or LG3. Some agents comprise or consist of fragments of any of the above peptides. Some such agents contain a segment of at least 5, 6, 7, 8, 9, 10, 11, 12, 13, 14, 15, 20, 50, 100, 150 amino acids within a specified peptide. Some such agents contain no more than 150, 100, 50, or 20 contiguous residues within a specified peptide. For example, some agents have 5-189, 5-100 or 5-50 contiguous amino acids within a specified peptide. Some agents have 10-100 or 10-50 contiguous amino acids from a specified peptide. Some agents have 20-100 or 20-50 contiguous amino acids from a specified peptide.

Some agents comprise or consist of peptides having amino acid sequences that are variants of DV or LG3 and or/any other peptide listed above. Such agents can be peptides, typically having at least 85, 90, 95 or 99% sequence identity with DV or LG3 (or other peptide listed above) over a comparison window of at 10, 25, 50, 100, 200, 300, 400, 500, 600, 700 or 728 amino acids present in both sequences being compared (not including gaps or residues aligned with gaps). The comparison window is preferably within DV and/or LG3. Such peptides preferably have a total length of no more than 800, 700, 600, 500, 400, 300, 200, 100, 50, 20 or 10 amino acids. Some agents have 20-728, 20-500, 20-189, 20-100 or 20-50 amino acids in total. Variants can be species, allelic or induced variants of DV or LG3. Induced variants can include non-natural or natural amino acids at positions differing from DV or LG3. Amino acid substitutions can be conservative or non-conservative.

Sequence variants likely to have conserved structure and/or function can be identified by conventional methods. For example, PFam and other domain identification tools can be used to identify amino acids required for the proper folding of protein domains, such as the Laminin G-like (LG) domain or the EGF-like domain. Conserved or conservatively substituted hydrophobic residues found in protein domains that have the same structure but differ in function are often involved in determining secondary and tertiary structure. Multi-sequence alignment tools can be used to identify conserved amino acids in protein domains having related function. Amino acid residues conserved in functionally related domains but not identified as critical for the proper folding of the domain are more likely to relate to the function of the protein domain. Structural prediction tools such as CABS, ESyPred3D, HHpred, ROBETTA, and WHAT IF can be used to model the structure of protein domains, allowing the identification of amino acids located at the surface of a protein domain. Conserved amino acids located at the surface of a protein domain are often involved in protein-protein interactions. In general, variation of amino acids at positions that are neither structurally nor functionally conserved typically results in functional variants; variation of amino acids at positions that are structurally and/or functionally conserved is more constrained, but is sometimes still permissible (e.g., conservative substitutions, and certain non-conservative substitutions may be permissible). The LG3 domain of perlecan DV is a member of the Lamanin_G₁ family (PFam designation PF00054) and contains two DGR sequences forming a binding motif for α2β1 integrin. Perlecan peptides including the two DGR sequences, optionally with some or all intervening amino acids, and with or without additional flanking perlecan sequences are provided, as are agents, comprising or consisting of such peptides. The sequences of DV from different organisms (e.g., human, mouse, rhesus monkey, horse, cow, chicken, and/or zebrafish) can be aligned to reveal structurally and functionally conserved residues. In addition, the LG3 domain can be structurally modeled, e.g., using the structure of pentraxin (see Beckmann et al. (1998), J. Mol. Biol. 275:725-730). The EGF-like domains of perlecan DV are members of the PFam EGF Clan CL0001 and can be structurally modeled based on known EGF-like domain structures. See Hohenester & Engel (2002), Matrix Biol. 21:115-128. Accordingly, using conventional sequence analysis tools and screening methods disclosed in the present application, DV and particularly LG3 sequence variants having conserved structure and function can be identified and tested for function.

Any of the peptides can be natural peptides or can be subject of modifications at the C or N-terminus or side chains.

Some agents include only a peptide segment of DV or LG3 or a peptide having a high degree of sequence identity (e.g., at least 85, 90, 95 or 99% with Swiss-Prot accession number P98160 (SEQ ID NO: 1)) over the length of the peptide. Other agents include an auxiliary molecule that can be a peptide or non-peptide. Such auxiliary molecules can serve as tags for purposes of identification or to improve pharmokinetics or to improve passage across the blood brain barrier. Examples of peptides that facilitate passage across the blood brain barrier include tat derived from HIV (Vives et al., 1997, J. Biol. Chem. 272:16010; Nagahara et al., 1998, Nat. Med. 4:1449) (e.g., YGRKKRRQRRR (SEQ ID NO: 2)), antennapedia from Drosophila (Dcrossi et al., 1994, J. Biol. Chem. 261:10444), VP22 from herpes simplex virus (Elliot and D'Hare, 1997, Cell 88:223-233), complementarity-determining regions (CDR) 2 and 3 of anti-DNA antibodies (Avrameas et al., 1998, Proc. Natl. Acad. Sci. U.S.A., 95:5601-5606), 70 kDa heat shock protein (Fujihara, 1999, EMBO J. 18:411-419) and transportan (Pooga et al., 1998, FASEB J. 12:67-77).

Some agents lack a peptide segment including residues 3464-3707 of Swiss-Prot accession number P98160. Some agents lack any subsegment of 5, 10, 20 or 50 contiguous residues between residues 3464 and 3707 of Swiss-Prot accession number P98160. Some agents lack any contiguous segment of at least 5, 10, 20 or 50 residues outside DV. Some agents lack any contiguous segment of at least 5, 10, 20 or 50 residues outside LG3.

Agents of the disclosure in particular specifically bind to α2 integrin usually when α2 integrin is complexed with β1 integrin. Agents of the disclosure in particular compete with DV and/or LG3 for binding to α2 integrin, again usually tested when complexed with β1 integrin.

### IV. Pharmaceutical Formulations and Methods of Administration

A. Pharmaceutical Formulations

In some embodiments of the present disclosure, the agents are included a pharmaceutical formulation. For example, where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The active compositions of the present disclosure may include classic pharmaceutical preparations. One will generally desire to employ appropriate salts and buffers to render agents stable and allow for uptake by target cells. Aqueous compositions of the present disclosure comprise an effective amount of the agent(s), dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Examples include phosphate buffered saline and buffered solutions of sodium lactate or sodium citrate with or without protein carrier. The use of such media and agents for pharmaceutically active substances is well known in the art. Except insofar as any conventional media or agent is incompatible with the present disclosure, its use in therapeutic compositions is contemplated. Supplementary active ingredients also can be incorporated into the compositions.

Agents of the disclosure are often administered as compositions comprising an active therapeutic agent and a variety of other pharmaceutically acceptable components. See Remington's Pharmaceutical Science (15th ed., Mack Publishing Company, Easton, Pa., 1980). The particular formulation employed depends on the intended mode of administration and the therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to negatively impact the biological activity of the combination. Examples of such diluents include, but are not limited to, distilled water, physiological phosphate-buffered saline, Ringer's solution, dextrose solution, and Hank's solution. In addition, the pharmaceutical composition or formulation may also include other carriers, adjuvants, or nontoxic, nontherapeutic, nonimmunogenic stabilizers, and the like.

Pharmaceutical compositions can also include large, slowly metabolized macromolecules such as proteins, polysaccharides such as chitosan, polylactic acids, polyglycolic acids, copolymers (such as latex functionalized Sepharose® beads, agarose, cellulose, and the like), polymeric amino acids, amino acid copolymers, and lipid aggregates (such as oil droplets or liposomes).

For parenteral administration, agents of the disclosure can be administered as injectable dosages of a solution or suspension of the substance in a physiologically acceptable diluent with a pharmaceutical carrier that can be a sterile liquid such as water, oils, saline, glycerol, or ethanol. Parenteral compositions for human administration are sterile, substantially isotonic, and made under GMP conditions. Additionally, auxiliary substances, such as wetting or emulsifying agents, surfactants, pH buffering substances, and the like, can be present in compositions. Other components of pharmaceutical compositions are those of petroleum, animal, vegetable, or synthetic origin, for example, peanut oil, soybean oil, and mineral oil. In general, glycols, such as propylene glycol or polyethylene glycol, are preferred liquid carriers, particularly for injectable solutions. Antibodies can be administered in the form of a depot injection or implant preparation that can be formulated in such a manner as to permit a sustained release of the active ingredient. An exemplary composition comprises monoclonal antibody at 5 mg/mL, formulated in aqueous buffer containing 50 mM L-histidine (optional), 150 mM NaCl, adjusted to a suitable pH with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or microparticles such as polylactide, polyglycolide, or copolymer for enhanced adjuvant effect, as discussed above (see Langer, Science, 249:1527 33 (1990) and Hanes et al., Advanced Drug Delivery Reviews, 28:97-119 (1997). The agents described herein can be administered in the form of a depot injection or implant preparation that can be formulated in such a manner as to permit a sustained or pulsatile release of the active ingredient.

Additional formulations suitable for other modes of administration include oral, intranasal, and pulmonary formulations, suppositories, and transdermal applications. Intranasal delivery is particularly useful for delivering peptides to the brain. Peptides can be formulated, for example, in sterile water, as a nasal spray. For suppositories, binders and carriers include, for example, polyalkylene glycols or triglycerides; such suppositories can be formed from mixtures containing the active ingredient in the range of about 0.5% to about 10%, or about 1% to about 2%. Oral formulations can include excipients such as pharmaceutical grades of mannitol, lactose, starch, magnesium stearate, sodium saccharine, cellulose, and magnesium carbonate. These compositions typically take the form of solutions, suspensions, tablets, pills, capsules, sustained release formulations or powders and contain about 10% to about 95% of active ingredient, or about 25% to about 70%.

The term “unit dose” refers to physically discrete units suitable for use in a subject, each unit containing a predetermined quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual. The agents of the present disclosure may be administered directly into animals, or alternatively, administered to cells that are subsequently administered to animals.

B. Methods of Administration

In prophylactic applications, pharmaceutical compositions or medicaments are administered to a patient susceptible to, or otherwise at risk of neurological injury, in a regime (i.e., dose, frequency, route of delivery) sufficient to at least reduce the risk, lessen the severity, or delay the onset of the injury, including biochemical, histological and/or behavioral symptoms of the injury, its complications and intermediate pathological phenotypes presenting during development of the injury.

Routes include intravenous, intraarterial, intracranial, intraperitoneal, intraosseous, intrathecal, subcutaneous, buccal (e.g., film), oral, intranasal (spray), aerosolized inhalation, any of which may be combined with a time-released formulation, such those using porous and resorbable nanostructures.

In therapeutic applications, compositions or medicaments are administered to a patient suspected of, or already suffering from such an injury in a regime (dose, frequency, route) sufficient to reduce, or at least slow deterioration of the symptoms of the disease (biochemical, histological, and/or behavioral), including its complications and intermediate pathological symptoms.

An amount adequate to accomplish therapeutic or prophylactic treatment is defined as a therapeutically- or prophylactically effective dose. In therapeutic regimes, the agent is usually administered at intervals until symptoms of the disease disappear or significantly decrease. Optionally administration can be continued to prevent recurrence. In prophylactic regimes, agents are also usually administered at intervals, in some instances for the rest of a patient's life. Treatment can be monitored by assaying levels of administered agent, or by monitoring the response of the patient.

Effective doses of the compositions of the present disclosure, for the treatment of the above-described conditions vary depending upon many different factors, including means of administration, target site, physiological state of the patient, whether the patient is human or an animal, other medications administered, and whether treatment is prophylactic or therapeutic. Usually, the patient is a human; nonhuman mammals, including transgenic mammals, can also be treated. Treatment dosages are typically titrated to optimize safety and efficacy.

Dosages of range from about 0.0001 to about 100 mg/kg, and more usually about 0.01 to about 20 mg/kg, of the host body weight. For example, dosages can be about 1 mg/kg body weight or about 20 mg/kg body weight or within the range of about 1 to about 10 mg/kg or 1-5 mg/kg. An exemplary treatment regime entails administration twice per day (such as every 12 hours), once per day, week, every two weeks or once a month or once every 3 to 6 months.

Agents of the disclosure can be administered by parenteral, topical, intravenous, oral, buccal (film), subcutaneous, intrathecal, intracranial, intraarterial, intracranial, intraperitoneal, intraosseous, intranasal, or intramuscular means for prophylactic and/or therapeutic treatment. In some methods, agents are injected directly into a particular tissue where deposits have accumulated, for example, intracranial injection. In some methods, intramuscular injection or intravenous infusion are employed for the administration of antibody. In some methods, particular therapeutic agents are injected directly into the cranium. In some methods, agents are administered as a sustained release composition or device.

With respect to whole organ/tissue transplantation, in addition to administering agents of the present disclosure to a subject, either before, at the time of, or after transplant, it is also contemplated that the agents could be used to treat, preserve, stabilize or otherwise benefit transplant materials per se. This could take the form of bathing or incubating the tissue with the agent, such as in solution in a “tissue/organ bath,” or it might involve infusing the agent into the tissue or organ through the tissue/organ's resident vasculature of by injection into the tissue/organ itself. Depending on the mode of administration, the expected duration of viability of the organ, and the urgency of the recipient, the incubation/treatment times may vary. Such tissue/organ treatments may also be used in combination with administration of the presently disclosed agents to the subject, as well as other standard pre- and/or post-transplant treatments.

TABLE 1
Proposed Recombinant Perlecan LG3 Proposed Therapeutic
Doses by Route of Administration*⁺

| | | |
|---|---|---|
| IV (intravenous): | 0.5-3 | mg/kg |
| IP (intraperitoneal): | 1.5-6 | mg/kg |
| IA (intraarterial): | 0.5-3 | mg/kg |
| IT (intrathecal): | 0.01-1 | mg/kg |
| IO (intraosseous): | 0.5-3 | mg/kg |
| IC (intracranial): | 0.01-1.5 | mg/kg |
| SC (Subcutaneous): | 1.5-6 | mg/kg |
| Oral (intrabuccal): | 0.5-6 | mg/kg |
| Intranasal: | 0.5-6 | mg/kg |

*Therapeutic agent may be safely administered to >10× dose presented above based on favorable safety profile.
⁺Possible veterinary application may be administered by reducing human dose by a factor of 2, 3, or 6 depending on species.

V. Combination Therapies

It is very common in the field of medicine to combine two or more therapeutic modalities. The following is a general discussion of therapies that may be used in conjunction with the therapies of the present disclosure.

To treat injuries or conditions using the methods and compositions of the present disclosure, one may contact a subject with an agent as disclosed herein and at least one other therapy. These therapies would be provided in a combined amount effective to achieve a reduction in one or more disease parameter. This process may involve contacting the subjects with the both agents/therapies at the same time, e.g., using a single composition or pharmacological formulation that includes both agents, or by contacting the subject with two distinct compositions or formulations, at the same time, wherein one composition includes the agent as disclosed herein and the other includes the other agent.

Alternatively, the agent as disclosed herein may precede or follow the other treatment by intervals ranging from minutes to weeks. One would generally ensure that a significant period of time did not expire between the time of each delivery, such that the therapies would still be able to exert an advantageously combined effect on the subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

Possible agents contemplated as combination therapies include immunomodulatory monoclonal antibodies targeting IL-6, CCLSR, CD3, CD6, a toll like receptor, or a receptor antagonists such as ATN-161 and IL1RA, or an NSAID, such as N-acteyl cysteine, acetylsalicylic acid, a nitric oxide synthase inhibitor, a COX-1, -2, and/or -3 inhibitor, or xanthine oxidase inhibitor.

It also is conceivable that more than one administration of either the peptide or the other therapy will be desired. Various combinations may be employed, where an agent of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Any therapy mentioned above in Section I could also be employed in a combination therapy with agents according to the present disclosure.

VI. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

The inventors designed an experiment to determine the effect of co-administering LG3 and tissue plasminogen activator (rtPA or tPA) with LG3. tPA is the only drug currently approved for the treatment of stroke. Its function is as a thrombolytic “clot buster” but does not on its own have any neuroprotective or neuroreparative effect. Importantly, tPA offers a narrow therapeutic window post-stroke to avoid hemorrhagic risk and possible death. This is because tPA reduces endogenous clotting activities and may weaken the vessels of the brain and the blood-brain barrier.

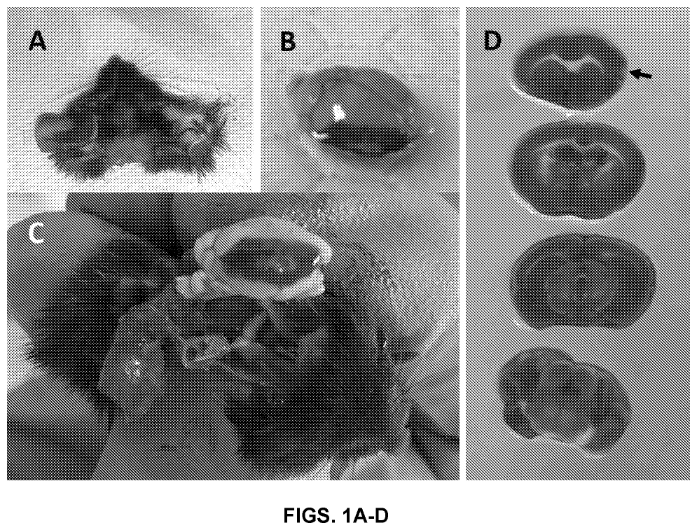

One of 10 mice that underwent tandem CCA-MCA 60 min occlusion with coadministration of tPA (10 mg/kg, IV) and LG3 (6 mg/kg, IP) at time of reperfusion was observed at PSD3 to have experienced a major brain bleed (FIGS. 1A-D) at some point following the end of the surgery and wound closure. This observation was unique among the ˜200 mice that have undergone CCA-MCA stroke in Stream's screening studies (none of which were administered tPA prior to this N=10 study). Following humane euthanasia and post-mortem decapitation, the researcher began to make an incision along the skin above the sagittal suture when they noticed significant amounts of coagulated blood. No attempt to measure the volume of coagulated blood was made, but coagulated blood at an estimated 1-3 mm thickness was present over the entire skull and in significant amounts at the skin/cranial interface under the ears of the mouse (FIG. 1A). Upon cleaning and opening the skull, coagulated blood was observed in an ovoid manner along the right midline of the brain (FIG. 1C). The epicenter of the bleed was directly aligned with the MCA at the point of occlusion. When sliced and stained with TTC, only a very minor infarct (arrow) was present around a small area of disrupted tissue (FIG. 1D).

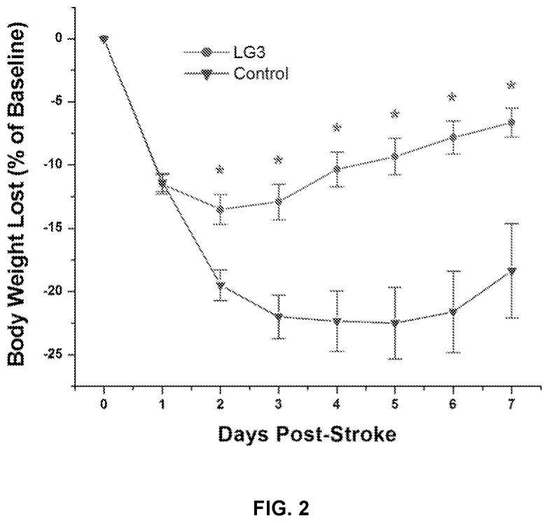

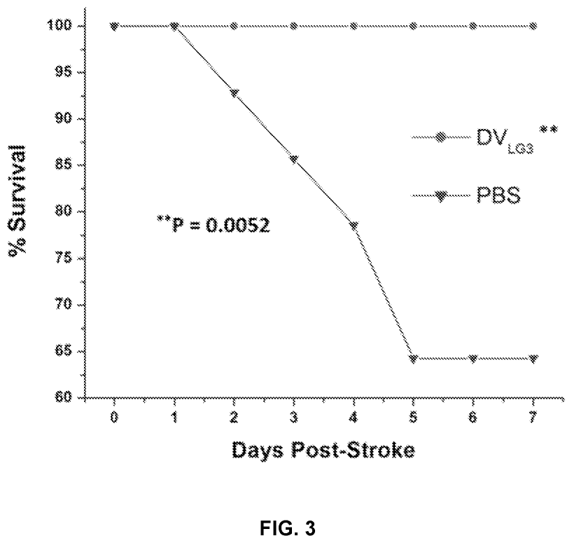

The subject showed no signs of distress of injury prior to euthanasia. The preoperative weight was 29 g and it was measured again at 29 g on PSD3 at the time of euthanasia. The subject was active and avoided handling and moved/fought with the vigor of a naïve mouse. The amount of blood and presence on and around the brain would suggest a mortal or debilitating injury, yet the slices show that the brain was largely spared from injury. It should be noted that the extent of injury observed here is in alignment with known values for 60-min CCA-MCA with 6 mg/kg LG3 treatment following occlusion (FIGS. 2-3). That implies that the brain was spared from hemorrhage and exacerbation of prior stroke injury that could have resulted from such an event. Thus, it appears likely that the tPA had caused the animal to hemorrhage, but LG3 treatment saved the animal's brain from damage. This is consistent with unpublished data showing that PDV and LG3 also tighten the blood-brain barrier junctions.

In addition to its potentially life-saving effects in the setting of acute ELVO, rhPDV and its fragments may confer protection from death to individuals suffering from aneurysmal hemorrhage or hemorrhage occurring during open and MIS brain surgery. It is contemplated that the protein therapeutic may be administered via IV drip during prolonged neurosurgical procedures targeting surgical exterpation of brain tumors or acutely via intrathecal injection for treatment of acute brain bleeds identified on MRI or PET scans.

### Example 2

An efficacy study was run investigating LG3 as a post-stroke therapeutic neuroprotective agent in mice that underwent 60-minute MCA Occlusion via intraluminal filament. This model is widely used and well accepted in stroke research and is recognized for producing extensive brain damage reflective of LVO in clinical populations. Significant mortality and functional deficits have been described in this model. Test drugs were administered three times via IP injection (6 mg/kg), once immediately following reperfusion/the end of stroke, then again at 48 hours and 96 hours post-reperfusion. Animal biometrics and activity were monitored throughout the study, with body weights recorded daily, and functional assessments at post-stroke day (PSD) 1, 3, 7. Infarct volume/stroke lesion size was quantified on PSD7.

LG3 was observed to exert a powerful effect on post-stroke recovery. LG3-treated subjects lost significantly less weight than vehicle-treated counterparts and displayed infarct volumes reduced by >60%. Analysis of body weight data revealed a profound change in the nature of weight loss in LG3-treated subjects: these subjects began gaining weight at PSD2-3 while vehicle-treated controls first demonstrated gain of weight at PSD5-6 (FIG. 2). Surprisingly, this observation was correlated with a marked improvement in function and a complete elimination of mortality (FIG. 3) associated with the stroke injury. Vehicle-treated controls experienced >33% mortality while zero mortalities (0%) were observed in LG3-treated cohorts. Data analysis revealed this difference was statistically significant (p=0.0052) based on our cohort size. This observation demonstrates that LG3 may exert powerful life-saving effects in stroke and other mortal neurotraumas.

### Example 3

Further studies have demonstrated LG3's potential life-saving effects across sex, age, and species. A study utilizing the transient filament MCAO stroke in aged (12 months) male and female rats treated with tPA prior to reperfusion was performed. The MCAO filament ischemic stroke model surgical procedure can lead to hemorrhage at the site of surgical MCA filament occlusion. Animals were treated with LG3 or vehicle (PBS) solution at the time of reperfusion, and again on PSD2 and PSD4. Functional outcomes were measured at baseline PSD1, 3, 7; and on PSD 7 all subjects were sacrificed and brains examined by a veterinary neuropathologist. For hemorrhage/safety and all other functional analyses, only animals without occlusion site hemorrhage (procedure artifact) were included.

LG3 treatment (6 mg/kg intraarterial administration at time of reperfusion) was observed by histology to eliminate the incidence of hemorrhage unrelated to the procedure compared to phosphate buffered saline treated counterparts. No hemorrhages (distinct from surgical occlusion site) were observed in any male or female subjects treated with LG3, compared to 29.6% of PBS vehicle-treated subjects (p=0.048). This observation further supports the notion that LG3 may exert significant therapeutic (life-saving) benefits in conditions where acute hemorrhage and brain trauma may occur.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A method of reducing the risk of mortality in a subject suffering from or at risk of neurological injury comprising administering to said subject a composition comprising perlecan domain V or a functional fragment thereof, wherein the condition is not a neurodegenerative disease.

2. The method of claim 1, wherein the functional fragment is LG3.

3. The method of claim 1, wherein the subject is suffering from a traumatic brain injury, such as caused by a concussive blast or blunt impact trauma.

4. The method of claim 1, wherein the subject is suffering from a stroke.

5. The method of claim 1, wherein the subject is at risk of a stroke, such as because said subject has previously suffered from a stroke, has atrial fibrillation, has diabetes, has hypertension, has cancer or has a viral infection, such as SARS-Cov-2 or a variant thereof.

6. The method of claim 4, wherein the stroke is an ischemic stroke, such as a large vessel occlusion.

7. The method of claim 6, wherein the large vessel occlusion is located in the anterior cerebral vasculature.

8. The method of claim 4, wherein the stroke is a hemorrhagic stroke.

9. The method claim 1, wherein administering comprises intraperitoneal administration, intravenous administration, intra-arterial administration, intrathecal administration, intracranial administration, intraosseous administration, intramuscular administration, subcutaneous administration, or oral (buccal) administration.

10. The method of claim 1, wherein the subject has received or is receiving a thrombolytic therapy, such as tissue plasminogen activator.

11. The method of claim 1, wherein said composition is administered more than once, such as 2, 3, 4, 5, 6, 7, 8, 9 or 10 times.

12. The method of claim 1, wherein said composition is administered twice daily, daily, every other day, every third day, twice a week, weekly, every other week, twice monthly or monthly.

13. The method of claim 1, further comprising administering to a second anti-stroke therapy.

14. The method of claim 1, wherein perlecan domain V is administered.

15. The method of claim 1, wherein LG3 is administered.

16. A method of reducing the risk of mortality in a subject suffering from a stroke and receiving a thrombolytic or embolytic therapy comprising administering to said subject perlecan domain V or a functional fragment thereof.

17. The method of claim 16, wherein the functional fragment is LG3.

18. The method of claim 16, wherein the stroke is an ischemic stroke.

19. The method of claim 18, wherein the ischemic stroke is a large vessel occlusion.

20. The method of claim 19, wherein the large vessel occlusion is located in the anterior cerebral vasculature.

21. The method of claim 16, wherein the stroke is a hemorrhagic stroke.

22. The method claim 16, wherein administering comprises intraperitoneal administration, intravenous administration, intra-arterial administration, intrathecal administration, intracranial administration, intraosseous administration, intramuscular administration, subcutaneous administration, or oral (buccal) administration.

23. The method of claim 16, wherein the thrombolytic therapy is tissue plasminogen activator.

24. The method of claim 16, wherein said composition is administered more than once, such as 2, 3, 4, 5, 6, 7, 8, 9 or 10 times.

25. The method of claim 16, wherein said composition is administered twice daily, daily, every other day, every third day, twice a week, weekly, every other week, twice monthly or monthly.

26. The method of claim 16, further comprising administering a further anti-stroke therapy.

27. The method of claim 16, wherein perlecan domain V is administered.

28. The method of claim 16, wherein of LG3 is administered.

29. The method of claim 16, wherein the thrombolytic therapy is administered within three hours of said stroke occurring.

30. The method of claim 16, wherein the thrombolytic therapy is administered more than three hours after said stroke occurring.

31. A method of reducing the risk of mortality in a subject suffering from a stroke comprising treating a subject with (a) perlecan domain V or a functional fragment thereof and (b) thrombectomy or embolectomy.

32. The method of claim 31, wherein the functional fragment is LG3.

33. The method of claim 31, wherein the stroke is an ischemic stroke.

34. The method of claim 33, wherein the ischemic stroke is a large vessel occlusion.

35. The method of claim 34, wherein the large vessel occlusion is located in the anterior cerebral vasculature.

36. The method of claim 31, wherein the stroke is a hemorrhagic stroke.

37. The method claim 31, wherein administering of perlecan domain V or a functional fragment thereof comprises intraperitoneal administration, intravenous administration, intra-arterial administration, intrathecal administration, intracranial administration, intraosseous administration, intramuscular administration, subcutaneous administration, or oral (buccal) administration.

38. The method of claim 31, wherein (b) is thrombectomy.

39. The method of claim 31, wherein (b) is embolectomy.

40. The method of claim 31, wherein perlecan domain V or a functional fragment thereof is administered more than once, such as 2, 3, 4, 5, 6, 7, 8, 9 or 10 times.

41. The method of claim 31, wherein said perlecan domain V or a functional fragment is administered twice daily, daily, every other day, every third day, twice a week, weekly, every other week, twice monthly or monthly.

42. The method of claim 36, further comprising administering a further anti-stroke therapy other than thrombectomy/embolectomy.

43. The method of claim 31, wherein perlecan domain V is administered.

44. The method of claim 31, wherein LG3 is administered.

45. The method of claim 31, wherein step (b) is performed within three hours of said stroke occurring.

46. The method of claim 31, wherein step (b) is administered more than three hours of said stroke occurring.

47. A method of treating, preserving, or stabilizing a transplant material comprising bathing, incubating, infusing, injecting or otherwise contacting the material with (a) perlecan domain V or a functional fragment thereof.


SEQUENCE LISTING (2 sequences)

SEQ ID NO: 1 - protein, 4391 aa, Homo sapiens
MGWRAAGALLLALLLHGRLLAVTHGLRAYDGLSLPEDIETVTASQMRWTHSYLSDDEDMLADSISGDDLGSGDLGSGDFQMVYFRALVNFTRSIEYSPQLEDAGSREFREVSEAVVDTLESEYLKIPGDQVVSVVFIKELDGWVFVELDVGSEGNADGAQIQEMLLRVISSGSVASYVTSPQGFQFRRLGTVPQFPRACTEAEFACHSYNECVALEYRCDRRPDCRDMSDELNCEEPVLGISPTFSLLVETTSLPPRPETTIMRQPPVTHAPQPLLPGSVRPLPCGPQEAACRNGHCIPRDYLCDGQEDCEDGSDELDCGPPPPCEPNEFPCGNGHCALKLWRCDGDFDCEDRTDEANCPTKRPEEVCGPTQFRCVSTNMCIPASFHCDEESDCPDRSDEFGCMPPQVVTPPRESIQASRGQTVTFTCVAIGVPTPIINWRLNWGHIPSHPRVTVTSEGGRGTLIIRDVKESDQGAYTCEAMNARGMVFGIPDGVLELVPQRGPCPDGHFYLEHSAACLPCFCFGITSVCQSTRRFRDQIRLRFDQPDDFKGVNVTMPAQPGTPPLSSTQLQIDPSLHEFQLVDLSRRFLVHDSFWALPEQFLGNKVDSYGGSLRYNVRYELARGMLEPVQRPDVVLMGAGYRLLSRGHTPTQPGALNQRQVQFSEEHWVHESGRPVQRAELLQVLQSLEAVLIQTVYNTKMASVGLSDIAMDTTVTHATSHGRAHSVEECRCPIGYSGLSCESCDAHFTRVPGGPYLGTCSGCNCNGHASSCDPVYGHCLNCQHNTEGPQCNKCKAGFFGDAMKATATSCRPCPCPYIDASRRFSDTCFLDTDGQATCDACAPGYTGRRCESCAPGYEGNPIQPGGKCRPVNQEIVRCDERGSMGTSGEACRCKNNVVGRLCNECADGSFHLSTRNPDGCLKCFCMGVSRHCTSSSWSRAQLHGASEEPGHFSLTNAASTHTTNEGIFSPTPGELGFSSFHRLLSGPYFWSLPSRFLGDKVTSYGGELRFTVTQRSQPGSTPLHGQPLVVLQGNNIILEHHVAQEPSPGQPSTFIVPFREQAWQRPDGQPATREHLLMALAGIDTLLIRASYAQQPAESRVSGISMDVAVPEETGQDPALEVEQCSCPPGYRGPSCQDCDTGYTRTPSGLYLGTCERCSCHGHSEACEPETGACQGCQHHTEGPRCEQCQPGYYGDAQRGTPQDCQLCPCYGDPAAGQAAHTCFLDTDGHPTCDACSPGHSGRHCERCAPGYYGNPSQGQPCQRDSQVPGPIGCNCDPQGSVSSQCDAAGQCQCKAQVEGLTCSHCRPHHFHLSASNPDGCLPCFCMGITQQCASSAYTRHLISTHFAPGDFQGFALVNPQRNSRLTGEFTVEPVPEGAQLSFGNFAQLGHESFYWQLPETYQGDKVAAYGGKLRYTLSYTAGPQGSPLSDPDVQITGNNIMLVASQPALQGPERRSYEIMFREEFWRRPDGQPATREHLLMALADLDELLIRATFSSVPLAASISAVSLEVAQPGPSNRPRALEVEECRCPPGYIGLSCQDCAPGYTRTGSGLYLGHCELCECNGHSDLCHPETGACSQCQHNAAGEFCELCAPGYYGDATAGTPEDCQPCACPLTNPENMFSRTCESLGAGGYRCTACEPGYTGQYCEQCGPGYVGNPSVQGGQCLPETNQAPLVVEVHPARSIVPQGGSHSLRCQVSGSPPHYFYWSREDGRPVPSGTQQRHQGSELHFPSVQPSDAGVYICTCRNLHQSNTSRAELLVTEAPSKPITVTVEEQRSQSVRPGADVTFICTAKSKSPAYTLVWTRLHNGKLPTRAMDFNGILTIRNVQLSDAGTYVCTGSNMFAMDQGTATLHVQASGTLSAPVVSIHPPQLTVQPGQLAEFRCSATGSPTPTLEWTGGPGGQLPAKAQIHGGILRLPAVEPTDQAQYLCRAHSSAGQQVARAVLHVHGGGGPRVQVSPERTQVHAGRTVRLYCRAAGVPSATITWRKEGGSLPPQARSERTDIATLLIPAITTADAGFYLCVATSPAGTAQARIQVVVLSASDASPPPVKIESSSPSVTEGQTLDLNCVVAGSAHAQVTWYRRGGSLPPHTQVHGSRLRLPQVSPADSGEYVCRVENGSGPKEASITVSVLHGTHSGPSYTPVPGSTRPIRIEPSSSHVAEGQTLDLNCVVPGQAHAQVTWHKRGGSLPARHQTHGSLLRLHQVTPADSGEYVCHVVGTSGPLEASVLVTIEASVIPGPIPPVRIESSSSTVAEGQTLDLSCVVAGQAHAQVTWYKRGGSLPARHQVRGSRLYIFQASPADAGQYVCRASNGMEASITVTVTGTQGANLAYPAGSTQPIRIEPSSSQVAEGQTLDLNCVVPGQSHAQVTWHKRGGSLPVRHQTHGSLLRLYQASPADSGEYVCRVLGSSVPLEASVLVTIEPAGSVPALGVTPTVRIESSSSQVAEGQTLDLNCLVAGQAHAQVTWHKRGGSLPARHQVHGSRLRLLQVTPADSGEYVCRVVGSSGTQEASVLVTIQQRLSGSHSQGVAYPVRIESSSASLANGHTLDLNCLVASQAPHTITWYKRGGSLPSRHQIVGSRLRIPQVTPADSGEYVCHVSNGAGSRETSLIVTIQGSGSSHVPSVSPPIRIESSSPTVVEGQTLDLNCVVARQPQAIITWYKRGGSLPSRHQTHGSHLRLHQMSVADSGEYVCRANNNIDALEASIVISVSPSAGSPSAPGSSMPIRIESSSSHVAEGETLDLNCVVPGQAHAQVTWHKRGGSLPSHHQTRGSRLRLHHVSPADSGEYVCRVMGSSGPLEASVLVTIEASGSSAVHVPAPGGAPPIRIEPSSSRVAEGQTLDLKCVVPGQAHAQVTWHKRGGNLPARHQVHGPLLRLNQVSPADSGEYSCQVTGSSGTLEASVLVTIEPSSPGPIPAPGLAQPIYIEASSSHVTEGQTLDLNCVVPGQAHAQVTWYKRGGSLPARHQTHGSQLRLHLVSPADSGEYVCRAASGPGPEQEASFTVTVPPSEGSSYRLRSPVISIDPPSSTVQQGQDASFKCLIHDGAAPISLEWKTRNQELEDNVHISPNGSIITIVGTRPSNHGTYRCVASNAYGVAQSVVNLSVHGPPTVSVLPEGPVWVKVGKAVTLECVSAGEPRSSARWTRISSTPAKLEQRTYGLMDSHAVLQISSAKPSDAGTYVCLAQNALGTAQKQVEVIVDTGAMAPGAPQVQAEEAELTVEAGHTATLRCSATGSPAPTIHWSKLRSPLPWQHRLEGDTLIIPRVAQQDSGQYICNATSPAGHAEATIILHVESPPYATTVPEHASVQAGETVQLQCLAHGTPPLTFQWSRVGSSLPGRATARNELLHFERAAPEDSGRYRCRVTNKVGSAEAFAQLLVQGPPGSLPATSIPAGSTPTVQVTPQLETKSIGASVEFHCAVPSDRGTQLRWFKEGGQLPPGHSVQDGVLRIQNLDQSCQGTYICQAHGPWGKAQASAQLVIQALPSVLINIRTSVQTVVVGHAVEFECLALGDPKPQVTWSKVGGHLRPGIVQSGGVVRIAHVELADAGQYRCTATNAAGTTQSHVLLLVQALPQISMPQEVRVPAGSAAVFPCIASGYPTPDISWSKLDGSLPPDSRLENNMLMLPSVRPQDAGTYVCTATNRQGKVKAFAHLQVPERVVPYFTQTPYSFLPLPTIKDAYRKFEIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRAQAGANTRPCPS

SEQ ID NO: 2 - protein, 11 aa, Artificial Sequence
  Synthetic Peptide
YGRKKRRQRRR